(19) United States
(12) Patent Application Publication
(10) Pub. No.: US 2022/0240903 A1
(43) Pub. Date: Aug. 4, 2022

(54) Device for collecting cervical cells for analysis

(71) Applicant: Kathleen Edmunds, Knoxville, TN (US)

(72) Inventor: Kathleen Edmunds, Knoxville, TN (US)

(21) Appl. No.: 17/300,002

(22) Filed: Feb. 2, 2021

(51) Int. Cl. A61B 10/02; A61B 10/00

(52) U.S. Cl. CPC A61B 10/02; A61B 2010/0216; A61B 2010/0074; A61B 10/0045

(57) ABSTRACT

A device for collecting cervical cells for analysis includes a thin saucer-shaped base portion having two opposite concave and convex side faces and having an outer edge which is oval in shape and elongate in form. In addition, the base portion is insertable into the vaginal canal of a female for cell-collecting purposes and includes a leading end which first enters the vaginal canal when the device is directed therein and an opposite trailing end. Furthermore, a plurality of bristles are joined to and extend away from the concave side of the base portion so that by inserting the device along the vaginal canal, the bristles of the device move across and collect cervical cells from the surface of the cervix.

### BACKGROUND OF THE INVENTION

This invention relates generally to means and methods for collecting cell tissue for analysis and relates, more particularly, to means and methods for collecting cervical cells for laboratory purposes.

The class of devices with which this invention is to be compared includes brush-including devices for collecting cervical cells for laboratory (e.g. pap smear) testing purposes. Such devices can include a long-handled device whose bristles extend longitudinally of the handle. One example of such a brush device is currently available from Wallach Surgical Devices of Trumbull, Conn. under the trade designation Papette.

It would be desirable to provide a sample-collecting device which possesses an improved capacity for collecting cervical cell samples for laboratory testing purposes.

Accordingly, it is an object of the present invention to provide a new and improved device for collecting cervical cells for laboratory purposes.

Another object of the present invention is to provide such a device which reduces the likelihood of user discomfort when used to collect cell samples.

Yet another object of the present invention is to provide such a device enabling the collection of cell samples by a patient (i.e. in a self-administered operation) or by a physician in a clinical setting.

Still another object of the present invention is to provide such a device which is uncomplicated in structure, yet effective in operation.

### SUMMARY OF THE INVENTION

This invention resides in a device for collecting cervical cells for analysis.

The device include a thin base portion having two opposite side faces and having an outer edge which is rounded in shape. In addition, the base portion is insertable into the vaginal canal of a female for cell-collecting purposes and includes a leading end which first enters the vaginal canal when the device is directed therein and an opposite trailing end. Furthermore, a plurality of bristles are joined to and extend away from one of the side faces of the base portion and substantially normal to the longitudinal axis of the base portion so that upon insertion of the base portion for cell-collecting purposes, the bristles of the device move across and collect cervical cells from the surface of the cervix.

### BRIEF DESCRIPTION OF THE DRAWINGS

FIG. 1 is a perspective view of an embodiment of a device within which features of the present invention are embodied, shown exploded.

FIG. 2 is a longitudinal cross-sectional view of the FIG. 1 device.

FIG. 3 is a top plan view of the FIG. 1 device, shown without the arrangement of brushes formed therein.

FIG. 4 is an end elevation view of the FIG. 1 device as seen generally from the right in FIG. 1.

FIG. 5 is a plan view of the FIG. 1 device wherein the tension member is shown in a gathered condition within the device.

FIG. 6 is a plan view of the FIG. 1 device as shown in FIG. 5, but showing the tension member in an extended condition.

### DETAILED DESCRIPTION OF AN ILLUSTRATIVE EMBODIMENT

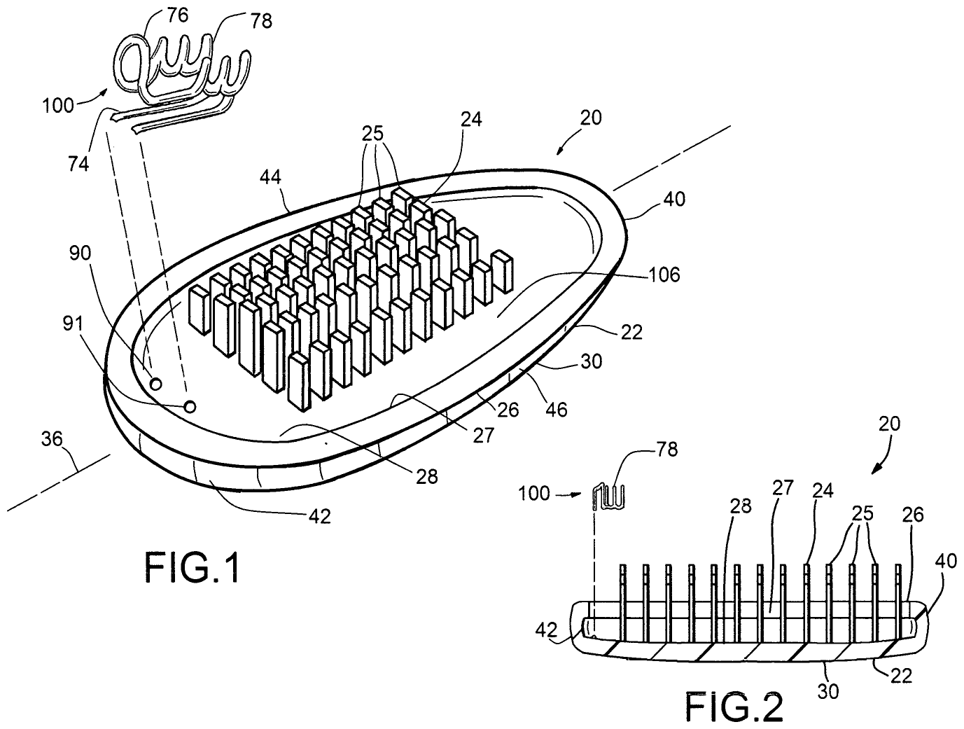

Turning now to the drawings in greater detail and considering first FIG. 1, there is illustrated an embodiment, generally indicated 20, of a sample, or cell, collecting device within which features of the present invention are embodied. The device 20 is preferably of one-piece (e.g. molded) construction and includes a base, or impermeable, portion 22 and a brush-including portion 24 having an arrangement of bristles 25 which are integrally formed (e.g. molded) into one side face of the base portion 22. As will be apparent herein, the device 20 is insertable along the vaginal canal of a female from whom cell samples are desired to be collected so that the bristles 25 move across the opening of the cervix. Such a movement of the device 20 through the vaginal canal effects the collection of cell samples from the surface of the cervix by the tips of the bristles 25.

In addition, the base portion 22 is shaped and sized to facilitate the insertion of the device 20 along the vaginal canal and to facilitate the subsequent removal of the device 20 from the vaginal canal following collection of the sample cells. The device 20 can be utilized by a patient for collection of cell samples in a self-administered process or, in the alternative, from a patient by a physician in a clinical setting.

With reference to FIGS. 1-4, the base portion 22 of the device 20 is saucer-shaped in form having a relatively shallow, concave side face 28 and an opposite convex side face 30. In addition, the base portion 22 is relatively thin as measured between the side faces 28, 30. Moreover, the base portion 22 is substantially inflexible in nature so as to provide a degree of stiffness or rigidity to the device 20 when inserted into and along the vaginal canal for sample-collecting purposes. Preferably, the base portion 22 is molded with integral ribs (or regions of increased thickness) which extend longitudinally along the concave side face 28 of the base portion 22. As will be apparent herein, the stiffness or rigidity of the base portion 22 is advantageous in that it helps to rigidify the device 20 when the device 20 is inserted through the vaginal canal.

To provide the base portion 22 with a desired degree of inflexibility, the base portion 22 can be constructed of any of a number of elastomeric materials, such as a relatively hard plastic (which can include silicon), or other classes of materials, such as a coated paper. However, it may be preferable to avoid latex as a choice of material for the base portion 22 due to the allergic reaction that some individuals have to that material.

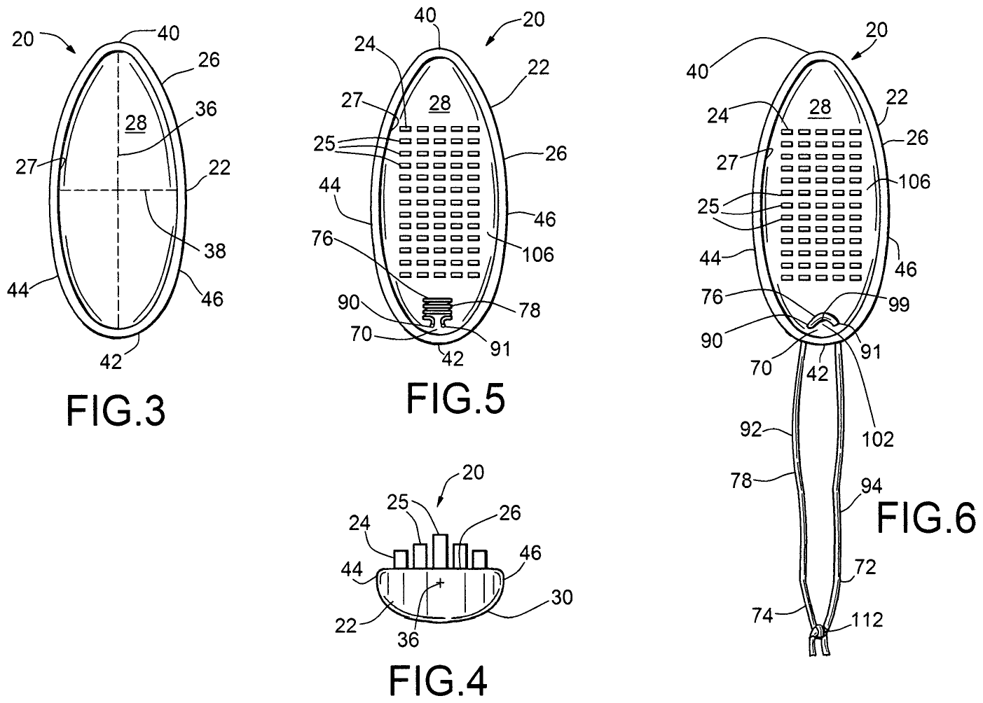

It is a feature of the device 20 that its base portion 22 has a rounded, or oval-shaped, outer edge 26 (defining an inwardly-directed lip 27 which encircles the concave side face 28. In this connection, the outer edge 26 of the base portion 22 of the depicted device 20 (as best shown in FIG. 3) is shaped to resemble an oval of markedly elongate form so that its maximum dimension (as measured along its major, or longitudinal, axis 36) is at least about 1.5 times the size of the minimum dimension (as measured along its minor axis 38).

While an oval, by definition, is symmetrical about at least one of its major or minor axes, the oval shape of the base portion 22 could be symmetrical about each of its major and minor axes 36, 38. However, the shape of the depicted base portion 22 somewhat resembles that of a teardrop having one (i.e. a leading) end 40 which is smaller, or more pointed in form, than the opposite (i.e. trailing) end 42 thereof.

Moreover and as used herein, the phrase “oval shape of markedly elongate form” is intended to connote that the outer edge 26 of the base portion 22 has an appearance which is distinctively elongate, and such a distinctively elongate appearance is described herein in structural terms by way of the stated requirement that the maximum diameter of the base portion 22 as measured along its major axis 36 is at least 1.5 times the size of the minimum dimension of the base portion 22 as measured along its minor axis 38.

The device 20, and more specifically, the base portion 22, is intended to be inserted endwise, or lengthwise, through the vaginal canal as the base portion 22 is directed and moved lengthwise (i.e. in a direction parallel to the major axis 36) through the canal. Moreover and for present purposes, the opposite sides of the base portion 22 which extend between the leading and trailing ends 40 and 42 are indicated 44 and 46 in FIGS. 1-4.

With reference again to FIGS. 1-3, the bristles 25 of the brush-including portion 24 of the device 20 are joined to and extend away from the concave side face 28 of the base portion 22. More specifically, the brushes 25 are integrally formed (e.g. molded) with the base portion 22 of the device 20 so that each bristle 25 extends away from one side face of the base portion 22 in a direction which is substantially normal to the longitudinal axis 36 of the base portion 22 and are arranged in rows and columns along the length of the base portion 22 and so that the front and back of each bristle 25 of the brush-including portion 24 face along the longitudinal axis 36 of the base portion 22. Furthermore and although each bristle 25 of the brush-including portion 24 is relatively thin as viewed from either the front, the back or the sides of the bristle 25, the side-to-side dimension of each bristle 25 is slightly larger than the front-to-back dimension of the bristle 25 so that the bristles 25 are more easily bent (along the length thereof) from front-to-back than from side-to-side. As will be apparent herein, such a front-to-back bendability of the bristles 25 renders the device 20 and, more particularly, the brush-including portion 24, relatively soft when inserted (longitudinally) along the vaginal canal. Furthermore and as best shown in FIG. 4, the bristles 25 of the brush-including portion 24 are slightly longer in the middle of the brush-including portion 24 than they are along the sides of the base portion 25. In other words, the row of brushes 25 which extend along, or adjacent, the longitudinal axis 36 of the base portion 22 are slightly longer than those in the rows of bristles 25 which extend along the sides 44, 46 of the base portion 22.

It is also a feature of the device 20 that it is provided with a tension member, generally indicated 100 in FIGS. 1 and 2, which is intended to aid the removal of the device 20 following a sample-collecting procedure yet remains, for the most part, arranged against the concave side face 28 of the base portion 22 and adjacent the trailing end 42 thereof until pulled upon during a device removal process. In this connection and with reference to FIGS. 5 and 6, the base portion 22 is provided with a pair of through-openings 90, 91 which extend between the concave and convex side faces 28, 30 thereof and which are disposed in a side-by-side-relationship adjacent the trailing end 42 of the base portion 22. In addition, the through-openings 90, 91 are spaced relatively close to one another and are separated from one another by a region, indicated 70 in FIG. 5, of the base portion 22.

With reference again to FIGS. 1, 5 and 6, the tension member 100 has two opposite ends 74, 76 and a major portion 78 which extends between the ends 74, 76. One of the ends 74 extends out of the base portion 22 through the through-openings 90, 91 and is accessible to a user, and the other of the ends 76 is prevented from exiting the base portion 22 when the one end 74 is pulled upon by user. Meanwhile, the major portion 78 of the tension member 100 is positioned within the interior, indicated 106, of the concave side face 28 of the base portion 22 adjacent the trailing end 42 of the device 20. As will be apparent herein, by pulling upon the one end 74 of the two opposite ends 74, 76 of the tension member 100 during a device removal process withdraws the tension member 100 from the interior 106 of the concave side face 28 to a fully extended condition, as best shown in FIG. 6, before the remainder of the device 20 is withdrawn from the user.

The tension member 100 of the depicted device 20 is in the form of a cord, or string, 72 which is comprised of a material (e.g. cotton) which is flexible in nature and has two opposite end portions 92, 94 (FIG. 6) which are folded back upon themselves so that one end 74 of the tension member 100 is provided by the two end portions 92, 94 positioned alongside one another (and tied in a knot 112 external of the base portion 22) and so that the other end 76 of the tension member 100 is provided by a looped mid-portion, indicated 99, of the cord 72.

During assembly of the device 20, the looped mid-portion 99 of the cord 100 can be secured, as with a stitch 102 (FIG. 6) of thread, to a fragment of the base portion 22 positioned adjacent the trailing end 42 thereof, and the two ends 92, 94 of the cord 72 are directed endwise for a short distance (e.g. less than about 0.5 inches) through the through-openings 90, 91 from the interior of the concave side face 28 and tied in a knot 112 external, or outside, of the interior of the concave side face 28 of the base portion 22. As will apparent herein, the knot 112 provides a user (e.g. either a patient or a physician) with a pull tab capable of being grasped (i.e. by a user) for purposes of removing the device 20 following use, and the looped mid-portion 99 (FIG. 6)—when the tension member 100 is pulled from the gathered condition, as best shown in FIG. 5, to a fully extended condition, as best shown in FIG. 6, acts upon the region, indicated 70 in FIG. 5, of the base portion 22 disposed between the through openings 90, 91 so that the device 20 is drawn from the vaginal canal by way of the tension member 100. It follows that the end 76 of the tension member 100 is thereby prevented from exiting the interior 106 of the concave side face 28 of the base portion 22 by the region 70.

It is another feature of the device 20 that the major portion 78 of the length of the tension member 100 remains positioned against the concave side face 28 of the base portion 22 and within the interior 106 until such time as it is desired to remove the device 20 following a sample collecting procedure. Preferably, the major portion 78 of the tension member 100 remains positioned in a gathered condition adjacent the concave side face 28, as depicted in FIG. 5. Such a gathered condition could take the form of, for example, a looped, folded, wound or linear arrangement to reduce the likelihood that the major portion 78 of the tension member 100 will become knotted when pulled (by way of the knot 112) to the fully-extended condition of FIG. 6 through the through-openings 90, 91, but the gathered condition of the major portion 78 could take an alternative form.

Because the trailing end 42 of the base portion 22 has been described above as being the end of the base portion 22 which follows the leading end 40 of the base portion 22 into the vaginal canal during a sample-collecting operation, the trailing edge 42 is the first end of the base portion 22 which exits the vaginal canal when withdrawn from the user. This being the case, the knot 112 provides a visual indication to the user as to which end of the base portion 22 corresponds with the trailing end 42 thereof.

During a sample collecting procedure, the device 20 is inserted along the vaginal canal as the base portion 22 is directed endwise (i.e. leading end 40-first) into and through the vaginal canal. The relatively small size of the smallest dimension (i.e. as measured across the minor axis 38) of the oval-shaped base portion 22 in comparison to the size of the largest, or lengthwise, dimension (i.e. as measured across the major axis 36) of the base portion 22 facilitates the lengthwise movement of the device 20 along the canal without requiring that the base portion 22 be compressed across the smallest dimension during insertion of the device 20 during insertion or removal of the device 20. Moreover, the oval shape of the base member 22 reduces the likelihood of discomfort to the female from whom cervix cells are being collected.

As suggested earlier, the removal of the device 20 from the vaginal canal is facilitated by the tension member 100 which can be pulled upon by the user. In particular and as the knot 112 is pulled upon by a user, the major portion 78 of the tension member 100 is withdrawn from the interior of the base portion 22 by way of the through-openings 90, 91 so that the tension member 100 begins to lengthen, or extend, as it is pulled, or withdrawn, from the base portion 22 before the base portion 22 begins to move along the vaginal canal toward the opening thereof. When the tension member 100 has been pulled from the interior of the concave side face 106 by a sufficient distance, the looped mid-portion 99 of the tension member cord 72 engages the region 70 of the base portion 22 and is prevented from exiting the interior 106 of the concave side face 28 by the region 70 so that any continued pull upon the knot 112 effects the withdrawn of the device 20 along the vaginal canal.

It follows from the foregoing that until the device 20 is desired to be removed following a sample collecting procedure, the major portion 78 of the tension member 100 remains in a gathered condition within the interior 106 of the concave side face 28 of the base portion 22. Furthermore, the base portion 22 does not have to be squeezed or compressed from side-to-side in order to remove the device 20 from the canal.

As mentioned earlier and upon insertion of the device 20 through the vaginal canal and across the opening of the cervix, the tips of the bristles 25 collect cells from the surface of the cervix. If desired, a long-handled clamp (not shown) can be used to grasp the trailing end 42 of the base portion 22 and gently manipulate the device 20 (by, for example, alternately rotating the device 20 rightwardly and leftwardly about the longitudinal axis 38 of the base portion 22) in somewhat of a scrubbing action to increase the amount of cells collected by the tips of the bristles 25. Moreover and because the bristles 25 of the brush-including portion 24 are shaped to bend less easily from side-to-side rather than from front-to-back, the aforedescribed rightward and leftward movement of the device 20 about the longitudinal axis 38 of the device 20 renders the bristles 25 more abrasive, and thus more effective, for cell-collecting purposes than would be the case if the device 20 were simply moved longitudinally across the surface of the cervix.

Upon retraction, or removal, of the device 20 from the vaginal canal following a cell-collecting process, the cells which are collected upon the tips of the bristles 25 of the brush-including portion 24 can be rinsed from the device 20 in a suitable solution (and in a known manner) for laboratory (e.g. pap smear) testing purposes.

The device 20 can be constructed in any of a number of sizes to accommodate a range of vaginal sizes. For example, the base portion 22 can be sized so that it measures about 65 mm, 75 mm or 85 mm along its major axis 38 to accommodate vaginal canals of different sizes.

As mentioned earlier, the device 20 can be used by a patient upon herself to collect cell samples in a self-administered process or alternatively, by a physician who collects cell samples from a female patient in a clinical setting. It has been found that by simply leaving the device 20 in place across the cervix for a predetermined amount of time (e.g. about four hours) before removal effects the collection of sufficient cervical cell samples by the tips of the bristles 25 for analysis purposes.

It will be understood that numerous modifications and substitutions can be had to the aforedescribed embodiment 20 without departing from the spirit of the invention. Accordingly, the aforedescribed embodiment 20 is intended for the purpose of illustration and not as limitation.

1. A device for collecting cervical cells for analysis, said device comprising:
a thin base portion having two opposite side faces and having an outer edge which is rounded in shape, wherein the base portion being insertable into the vaginal canal of a female for cell-collecting purposes and includes a leading end which first enters the vaginal canal when the device is directed therein and an opposite trailing end; and
a plurality of bristles which are joined to and extend away from one of the side faces of the base portion so that upon insertion of the device for cell-collecting purposes, the bristles of the device move across and collect cervical cells from the surface of the cervix.

2. The device as defined in claim 1 wherein the side face of the base portion has a length as measured between the leading and trailing ends of the base portion, and the plurality of bristles are joined to said one side face of the base portion and extend over a substantial fraction of the length thereof.

3. The device as defined in claim 2 wherein the base portion has a longitudinal axis which extends between the leading and trailing ends thereof, and said one side face of the base portion has a width as measured transversely of the longitudinal axis of the base portion, and
the plurality of brushes extend over a substantial fraction of the width of said one side face.

4. The device as defined in claim 3 wherein each bristle of the plurality of bristles has a length, and the length of the bristles of the plurality of bristles is the greatest along a path which is traced along the longitudinal axis of the base portion.

5. The device as defined in claim 1 wherein the plurality of bristles and the base portion of the device are formed as a single unit.

6. The device as defined in claim 1 wherein the base portion is relatively stiff so as to resist compression of the base portion from side to side.

7. The device as defined in claim 1 wherein each bristle of the plurality of bristles has a width which is measured transversely of the base portion and a thickness which is measured longitudinally of the base portion, and the width of each bristle of the plurality of bristles is larger than the thickness thereof.

8. The device as defined in claim 1 wherein the base portion includes an inwardly-directed lip which extends around said one side face thereof.

9. The device as defined in 1 wherein the base portion defines a through-opening therein adjacent the trailing end thereof, and the device further comprises
a tension member facilitating the withdrawal of the device following use, the tension member having two opposite ends and a major portion which extends between the two opposite ends, the major portion of the tension member being positioned against said one side face of the base portion and adjacent the trailing end thereof, one end of the two opposite ends of the tension member protrudes through the through-opening defined in the base portion and is accessible to a user, and the other of the two opposite ends of the tension member is prevented from passing through said one side face through the through-opening so that by pulling upon the one end of the two opposite ends of the tension member during a device removal process withdraws the tension member from its position against said side face to a fully extended condition before the remainder of the device is withdrawn from the user.

10. The device as defined in claim 9 wherein the major portion of the tension member is gathered against said one side face and the plurality of bristles during insertion of the device during a cell-collecting operation.

11. The device as defined in claim 10 wherein the tension member is in the form of a cord having two opposite ends and wherein the cord is folded back upon itself so that one end of the tension member is provided by the two opposite ends of the cord and the other end of the tension member is provided by a looped mid-section of the cord; and
wherein the base portion includes a pair of through-openings which are arranged in a side-by-side relationship with one another and which extend between the side faces of the base portion, and wherein portions of the cord extend through both of the pair of through-openings so that by pulling upon the two opposite ends of the cord, the other end of the tension member is prevented from passing through the base portion by way the looped mid-portion of the cord.

12. The device as defined in claim 11 wherein the two opposite ends of the cord which extend through the pair of through-openings are tied together in a knot external of the base portion.

13. The device as defined in claim 11 wherein the cord is in the form of a cotton string.

14. An insertable device for collecting cervical cells for laboratory purposes, the device comprising:
a relatively thin, saucer-shaped base portion having two opposite concave and convex side faces and having an outer edge which is oval in shape and markedly elongate in form and has a longitudinal axis, wherein the concave side face has an interior and the base portion includes a leading end which first enters a vaginal canal when the device is inserted therein for cell-collecting purposes and an opposite trailing end, and the base portion defines an opening therein adjacent the trailing end thereof and an inwardly-facing lip which encircles the concave side face of the base portion;
a plurality of bristles which are joined to and extend away from the concave side face of the base portion along paths which are substantially normal to the longitudinal axis of the base portion so that upon insertion of the device past the cervix, the bristles of the device move across and collect cervical cells from the surface of the cervix; and
a tension member is attached to the base portion by way of the opening provided therein for facilitating the withdrawal of the device following collection of cervical cells.

15. The device as defined in claim 14 wherein the concave side face of the base portion has a length as measured between the leading and trailing ends of the base portion, and
the plurality of bristles are joined to the concave side face of the base portion and extend over a substantial fraction of the length thereof.

16. The device as defined in claim 15 wherein the concave side face of the base portion has a width as measured transversely of the longitudinal axis of the base portion, and
the plurality of brushes extend over a substantial fraction of the width of the concave side face.

17. The device as defined in claim 16 wherein each bristle of the plurality of bristles has a length, and the length of the bristles of the plurality of bristles is the greatest along a path which extends along the longitudinal axis thereof.

18. The device as defined in claim 14 wherein the plurality of bristles and the base portion of the device are molded as a single unit.

19. The device as defined in claim 14 wherein the base portion is relatively stiff so as to resist compression of the base portion from side to side.

20. The device as defined in claim 14 wherein each bristle of the plurality of bristles has a width which is measured transversely of the base portion and a thickness which is measured longitudinally of the base portion, and the width of each bristle of the plurality of bristles is larger than the thickness thereof.
